(19) United States
(12) Patent Application Publication
(10) Pub. No.: US 2012/0190092 A1
(43) Pub. Date: Jul. 26, 2012

(54) Processes And Systems For Enzymatically Isolating Lignin And Other Bioproducts From Herbaceous Plants

(75) Inventors: Percy Jaquess, Memphis, TN (US); Bernard Janse, Memphis, TN (US)

(73) Assignee: BUCKMAN LABORATORIES INTERNATIONAL, INC., Memphis, TN (US)

(21) Appl. No.: 13/351,285

(22) Filed: Jan. 17, 2012

Related U.S. Application Data
(60) Provisional application No. 61/435,492, filed on Jan. 24, 2011.

(51) Int. Cl. C07H 1/06; C12P 7/14; C12M 1/40; C12S 3/10; C07H 1/08

(52) U.S. Cl. 435/162; 435/276; 435/283.1; 435/289.1

(57) ABSTRACT

Methods for enzymatically isolating lignin and other bioproducts, such as fermentable sugars, from herbaceous plant materials, are described. The methods can provide improvements, such as increased product purity and reduced process energy requirements and product modifications and contamination. Systems for practicing the methods also are provided.

### BACKGROUND OF THE INVENTION

This application claims the benefit under 35 U.S.C. §119(e) of prior U.S. Provisional Patent Application No. 61/435,492, filed Jan. 24, 2011, which is incorporated in its entirety by reference herein.

The present invention relates to enzymatic isolation of lignin and other bioproducts from herbaceous plants, and particularly, to processes to enzymatically isolate lignin and sugars from lignocellulosic materials, and systems for conducting these processes.

A potentially plentiful source of biomass for the production of bioproducts is herbaceous lignocellulosic plant fiber. Some cellulose polysaccharides, for example, found in the plant fiber can be isolated from the lignin and degraded into fermentable sugars. These sugars can be fermented, for example, to produce alcohols (e.g., ethanol, butanol), organic acids (e.g., acetic acid, citric acid), and other products (e.g., other hydrocarbons, proteins). Lignin has been used in a variety of applications, including as an energy source when burned, and as a raw material or additive for various chemicals and compositions. As generally understood, however, the natural structure of lignocellulosic material can make the material very resistant to fractionation without resorting to harsh and/or energy intensive measures.

Lignocellulosic biomasses may contain, for example, about 35-50 wt % cellulose, about 15-35 wt % hemicellulose, and about 5-30 wt % lignin, or other proportions depending on its origin. Although cellulose, hemicellulose, and lignin often can be the major components of lignocellulosic biomass, there also can exist varying amounts of other materials present in both bound and unbound forms. These minor components can include, for example, proteins, uronic acids, acetic acid, ash, free sugars such as sucrose, soil, and foreign materials. Cellulose generally is a polymer of glucose. The glucose molecules can be joined by beta-1,4-glycosidic linkages. Hemicellulose is a polymer generally containing primarily 5-carbon sugars, such as xylose and arabinose, which can have some glucose and mannose dispersed throughout. Hemicellulose tends to form a polymer that interacts with cellulose and lignin in the plant wall, strengthening it. Lignin tends to help bind the cellulose-hemicellulose matrix while adding flexibility. The molecular structure of lignin polymers typically can be, for example, random and disorganized and can consist primarily of carbon ring structures (e.g., benzene rings with methoxyl, hydroxyl, and propyl groups) interconnected by polysaccharides. The lignin-hemicellulose matrix typically encases cellulose, and makes the material recalcitrant to digestion.

Treatment of lignocellulosic material to separate lignin from biopolymers in the biomass has been an actively researched field and a wide variety of thermal, mechanical, and chemical pretreatment approaches (and combinations thereof) have been investigated and reported. Harsh treatments have been used in the past, such as involving strong chemicals and/or intense heat processing, to separate lignin from other constituents of lignocellulosic materials. The separation of cellulose polysaccharides, for example, from the protective lignin in the herbaceous lignocellulosic plant fiber material in some prior processes has required high energy costs, or caused modified or contaminated products, or affected downstream processing thereof, or had other drawbacks.

Kraft or sulfite pulping processes, such as has been used in paper mills, removes lignin from the cellulose fibers of softwoods, hardwoods, or both, by pretreatment of pulp with concentrated acid (e.g., sulfuric acid), sodium sulfide, or salts of sulfuric acid, as a preliminary operation to papermaking. Treatment of lignocellulosic material with concentrated acid or concentrated alkali/alkaline agents can cause production of lignosulfonates, which are sulfur-contaminated lignin materials. Harsh acid treatments on lignocellulose material also can lead to the presence of inhibitors, which can complicate downstream processing of bioproducts and increase the cost of production due to entailed detoxification steps. Another prior technique for removing the lignin and exposing the cellulose has included, for example, use of high pressure steam, which can entail increased energy costs. Prior process practices for liberating lignin and sugars from cotton seed hulls, for example, has included treatment of the material with high temperature (e.g., >180° C.) and steam pressure, which requires significant energy. Combined use of concentrated acids/alkalis and steam treatments on lignocellulosic material also has been reported, which may amplify the indicated drawbacks. Another prior process has used irradiating devices to ionize the lignocellulosic biomass feedstock. Irradiation can alter the structure of the lignin component and its lignin derivatives. Other prior technologies have used recombinant polypeptides for enhancing release of other hydrolyzing enzymes. Use of recombinant polypeptides can involve higher materials costs and does not use natural agents. Prior practices also have used a continuous bioreactor process with a bioreactor film for continuous processing. The use of a bioreactor film can require extended processing times with inconsistent yields of components. Other prior processes have used live anaerobic cultures of organisms to perform the needed release tasks. Use of anaerobic live cultures can increase processing complexity and costs.

The present inventors have recognized that certain components and derivatives of components of lignocellulosic materials (e.g., cotton seed hulls), such as lignin and sugars, would be more useful as industrial raw materials if they can be isolated in purer forms without degrading the extracted lignins and sugars and with less waste by-products. The present investigators further have recognized a need for isolating lignin and sugars from herbaceous plant materials, such as cotton seed hulls, exclusively using natural, more environmentally-friendly, and recyclable active agents, with less energy requirements.

### SUMMARY OF THE INVENTION

A feature of this invention is to provide a method that isolates lignin and other bioproducts from herbaceous lignocellulosic plants using a total enzymatic process.

An additional feature of this invention is to provide a method that isolates lignin and sugars from cotton seed hulls using a total enzymatic process.

Another feature of this invention is to provide a method that isolates lignin and sugars from cotton seed hulls using a total enzymatic process without requiring high temperature processing, ionization irradiation processing, strong acids, strong alkaline agents, chelating agents, recombinant DNA, and/or live anaerobic microorganism cultures.

Additional features and advantages of the present invention will be set forth in part in the description which follows, and in part will be apparent from the description, or may be learned by practice of the present invention. The objectives and other advantages of the present invention will be realized and obtained by means of the elements and combinations particularly pointed out in the written description and appended claims.

To achieve these and other advantages and in accordance with the purposes of the present invention, as embodied and broadly described herein, the present invention, in part, relates to a method for isolating components of lignocellulosic material comprising comminuting lignocellulosic material to smaller particle sizes having an increased surface area. A mixture comprising the comminuted lignocellulosic material, at least one enzyme blend, and aqueous solution then is enzymatically digested in the absence of prior non-enzymatic lignin separation treatment of the lignocellulosic material, for enzymatic hydrolysis liberating at least one C6 and/or C5 sugar from an insoluble portion of the lignocellulosic material comprising lignin. At least a portion of the aqueous solution containing the at least one C6 and/or C5 sugar then is separated from the insoluble portion comprising lignin for recovery of either or both bioproducts.

The present invention further relates to a method for isolating components of lignocellulosic material comprising comminuting lignocellulosic material to smaller particle sizes having a first increased surface area, and then enzymatically digesting a first mixture comprising the comminuted lignocellulosic material, a first enzyme blend, and a first aqueous solution in the absence of any prior non-enzymatic lignin separation treatment of the lignocellulosic material, for a first enzymatic hydrolysis liberating at least one C6 and/or C5 sugar from a first insoluble portion of the lignocellulosic material comprising lignin. At least a portion of the first aqueous solution containing the at least one C6 and/or C5 sugar then is separated from the first insoluble portion, wherein the separated first insoluble portion then is comminuted to smaller particles sizes having a second increased surface area. A second mixture comprising the comminuted first insoluble portion, a second enzyme blend, and a second aqueous solution then is enzymatically digested for a second enzymatic hydrolysis liberating at least one C6 and/or C5 sugar from a second insoluble portion comprising lignin, and the second insoluble portion comprising lignin then is separated from the second aqueous solution.

It is to be understood that both the foregoing general description and the following detailed description are exemplary and explanatory only and are only intended to provide a further explanation of the present invention, as claimed.

As used herein, “lignocellulosic material” or “lignocellulose” refers to a plant cell wall material containing lignin and cellulose, and optionally also hemicellulose, in a matrix. Lignocellulosic material containing these constituents can be, but is not limited to, herbaceous material, agricultural residues, forestry residues, municipal solid wastes, recycled paper and waste paper (e.g., nonbleached paper), and pulp and paper mill residues (e.g., nonbleached pulp and mill residues).

“Lignin” refers to a complex biopolymer which is an integral part of the secondary cell walls of plants and some algae. Lignin fills spaces in the cell wall and between cellulose, hemicellulose, and, if present, pectin components. Lignin can be identified, for example, with CAS Number 9005-53-2.

“C6 and/or C5 sugar” refers to monosaccharides including, for example, hexose (“C6”) sugars (e.g., aldohexoses such as glucose, mannose, galactose, gulose, idose, talose, aldohexose, allose altrose; and ketohexoses such as psicose, fructose, sorbose, tagatose; or others, singly or in any combinations thereof), and/or pentose (“C5”) sugars (e.g., aldopentosess such as xylose, arabinose, ribose, lyxose; ketopentoses such as ribulose, xylulose; and others, singly or in any combinations thereof). Hexose is a monosaccharide with six carbon atoms, having the chemical formula C₆H₁₂O₆. Hexoses can be classified, for example, by a functional group, with aldohexoses having an aldehyde functional group at position 1, and ketohexoses having a ketone functional group at position 2. As known, 6-carbon aldose sugars can form cyclic hemiacetals, which can include a pyranose structure. In solution, open-chain forms and cyclic forms of 6-carbon aldose sugars can exist in equilibrium, or be present in other relative fractions to each other. A diagram included in the figures herein shows open chain and cyclic forms for D-glucose and D-mannose for sake of illustration only and not necessarily limitation (FIG. 4). As shown in the indicated figure, the numbering of carbons for the hexose is indicated with respect to the open chain form thereof (a similar carbon numbering system is used for the pentoses). Any puckered structure (e.g., chair conformation) in the cyclic rings shown in the figure provided in this respect are not illustrated in order to simplify the illustration, and these structures should be understood by persons skilled in the art. Pentose is a monosaccharide with five carbon atoms, having the chemical formula C₅H₁₀O₅. Pentose can be classified, for example, into two groups, with aldopentoses having an aldehyde functional group at position 1, and ketopentoses having a ketone functional group at position 2. As known, 5-carbon aldose sugars also can have cyclic hemiacetals forms, which can include a furanose structure. The hemiacetal cyclic forms of 5-carbon aldose sugars may spontaneously open and close, wherein mutorotation may occur.

“Glucan” refers to a polysaccharide of D-glucose monomers linked by glycosidic bonds. Glucan can refer to beta-glucans (e.g., cellulose, curdlan, laminarin, chrysolaminarin, lentinan, lichenin, pleuran, zymosan), alpha-glucan (e.g., dextran, glycogen pullulan, starch), either singly or in any combinations thereof.

“Enzymatic activity” refers to enzymatic hydrolytic activity unless indicated otherwise.

“Non-enzymatic lignin separation treatment” refers to any of a high temperature/pressure process, an ionization irradiation process, a strong acid treatment, a strong alkaline agent treatment, a chelating agent treatment, a recombinant DNA treatment, or a live anaerobic microorganism culture treatment, or any other treatment process that is not an enzyme-based hydrolytic process, which can cause or induce hydrolysis of lignocellulosic material and is harsh enough to separate lignin from C6 and/or C5 sugars of a lignocellulosic material in detectible amounts.

The accompanying drawings, which are incorporated in and constitute a part of this application, illustrate aspects of the present invention and together with the description, serve to explain the principles of the present invention.

### BRIEF DESCRIPTION OF THE DRAWINGS

FIG. 1 illustrates a process block diagram of a method of isolating lignin and other bioproducts from lignocellulosic material according to an example of the present invention.

FIG. 2 illustrates a process block diagram of a method of lignin and sugar isolation from lignocellulosic material, illustrated as cotton seed hulls, according to an example of the present invention.

FIG. 3 illustrates a process flow diagram of a method of lignin and sugar isolation from lignocellulosic material according to an example of the present invention.

FIG. 4 shows non-limiting illustrations of open chain and cyclic forms of D-glucose and D-mannose.

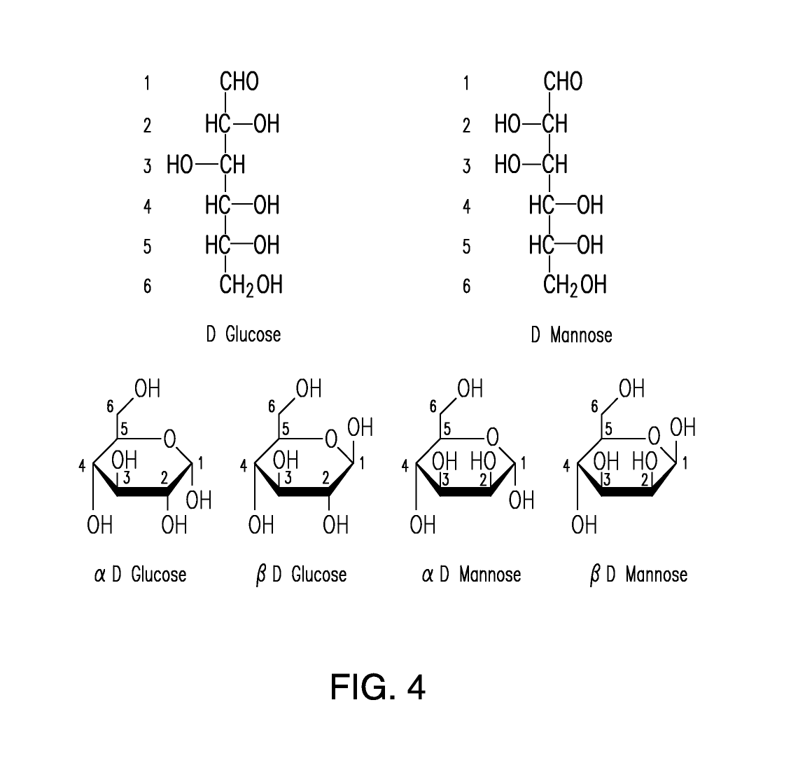

### DETAILED DESCRIPTION OF THE PRESENT INVENTION

The present invention provides methods of enzymatically purifying lignin from herbaceous plants with concomitant liberation of recoverable sugars and/or other bioproducts. The present methods can be total enzyme-based processes without need of any harsh non-enzymatic pre- or co-treatments of the lignocellulosic material which may degrade the quality of the bioproducts and/or otherwise complicate or increase costs of production. In the present methods, lignocellulosic feedstock can be pretreated by physically reducing the physical size of the feedstock particulate to increase its surface area and/or otherwise render the material more accessible for a subsequent enzymatic hydrolysis treatment of the cellulose molecules. In the enzymatic hydrolysis subsequently performed on the pretreated (resized) lignocellulosic material in methods of the present invention, the cellulose molecules can be broken down to liberate fermentable sugars and other smaller molecules, and are separated from the lignin, for their respective recoveries in higher purified forms than possible without the comminution pretreatment. For example, cellulose chains in the comminuted (resized) lignocellulosic material produced as an intermediate material in methods of the present invention can be more readily broken into C6 and/or C5 sugar molecules and isolated from lignin by use of enzymes alone. The comminution used in the present methods can be performed in a preferred manner which do not cause or induce hydrolysis of the lignocellulosic feedstock, and other than resizing the material, leaves the original constituents (e.g., lignin, cellulose) substantially or essentially completely intact for subsequent isolation processing. The present methods provide enzymatic hydrolysis reactions on the comminuted feedstock, wherein enzyme blends are used in one or multiple digestion stages. An overall strategy feasible with the present method is to remove (digest) as much polysaccharide (e.g., cellulose, xylan, hexose, and/or pentose polymers) present in the comminuted lignocellulosic feedstock as feasible and yield isolated lignin in an essentially intact form, and C6 and/or C5 sugars, for example, fermentable C6 and/or C5 sugars. Further, lignocellulosic materials can be enzymatically hydrolyzed at a relatively mild condition in methods of the present invention (e.g., about 50° C., pH of about 5, and at approximately atmospheric pressure, or other mild conditions). This can permit effective cellulose breakdown with reduced formation of undesirable by-products (e.g., product contaminants and reaction inhibitors) which can be associated with harsher hydrolysis conditions. Furthermore, as shown by results of experimental studies described in the examples herein, it has been found that even without including a harsh non-enzymatic pretreatment of the lignocellulosic feedstock (e.g., a strong acid treatment or treatment with milder acid at high temperature and pressure), that methods of the present invention can achieve a substantial enzymatic hydrolysis of the lignocellulosic material to provide highly purified lignin, fermentable sugars, or both.

Herbaceous plants can be a source of lignocellulosic material, which can be processed using methods of the present invention. The lignocellulosic material can be, for example, a wood material, a grass material, and/or a waste product derived from these materials, which contain lignin (e.g., at least 1 wt %, or at least 3 wt %, or at least 5 wt %, or at least 10 wt %, or at least 15 wt %, or at least 25 wt % lignin, on a dry solids basis). Lignocellulosic material can be grouped, for example, into categories of agricultural residues and waste (e.g., cotton seed hulls, grain hulls, sugarcane bagasse, corn stover, corn cobs, straw, switchgrass, leaves, stalks, shells, etc.), forestry products (e.g., softwoods, hardwoods, etc.), wood scraps (e.g., sawdust, wood chips, bark, etc.), papermill pulp, and waste paper or recycled paper (e.g., unbleached paper, newsprint), and other lignocellulosic materials, or any combinations thereof. In lignocellulosic material, carbohydrate polymers (e.g., cellulose and hemicelluloses) are tightly bound to lignin, wherein fermentable sugars or precursors thereof are trapped inside a matrix formed with the lignin and hemicellulose. The methods of the present invention can be directed to overcoming this barrier to the isolation and purification of lignin, and production of other useful bioproducts, from lignocellulosic material, in economical manners without degrading or contaminating the desired bioproducts and without requiring harsh non-enzymatic treatments of the lignocellulosic material.

In the present methods, the lignocellulosic material can be processed in the form of a particulate, and particle size can be important as it can directly effect the surface area available for interaction with enzymes used to treat the particles. In the methods of the present invention, particle-size reduction (i.e., comminution) is performed before enzymatic hydrolysis processing in order to provide higher purity products. While not desiring to be bound to a theory, the comminution before enzymatic digestion can increase the accessibility of enzymes to the lignocellulosic material by increasing the overall surface area of the material. This is thought to make it easier for enzyme hydrolytic activity to occur at exposed surfaces on the lignocellulosic material, wherein the celluloses can be more easily disconnected from the lignin. The liberated celluloses can be further enzymatically hydrolyzed to degrade them down into simple monosaccharides or other fermentable sugars. Further, the enzymatic hydrolysis can be conducted on the comminuted lignocellulosic particles, for example, in a mildly acidic, organic acid buffered aqueous solution under mild heating conditions at atmospheric pressure, reducing or avoiding the need for harsh chemicals or energy intensive operations.

With respect to general mechanisms, a blend of enzymes can be used in methods of the present invention in a single-stage or a multi-stage digestion of the lignocellulosic material. Six carbon (C6) and/or five carbon (C5) sugars are liberated from the biopolymer matrix of the herbaceous material (e.g., cotton seed hulls (CSH)). Lignin is left as a remainder biopolymer in a solids fraction which is not hydrolyzed. After digestion agitation is discontinued, the lignin aggregates within itself and easily precipitates out of solution as a precipitate, which can be further processed and/or recovered as finished product. When the hydrolysis is performed upon native herbaceous material that has not been sulfonated by way of chemical processing, as in enzymatic hydrolysis of methods of the present invention, the resulting aggregated lignin is uncontaminated with sulfur in the form of sulfonates. The liberated C6 and/or C5 sugars (e.g., glucose, mannose, xylose, arabinose, etc.) are solubilized or suspended in the hydrolysis solution and are available for recovery from the liquid fraction of the digested mixtures and for possible further use, if desired. After removal of lignin, the recovered C6 and/or C5 sugars in the removed sugar fraction or fractions can be, for example, directly converted to ethanol or other biochemical based products using fermentation methodology by means of introducing appropriate conversion organisms of choice, including those which will be recognized in the bioethanol fermentation industry. The lignin can be used as fuel source, as a raw material or an additive, and/or for other applications.

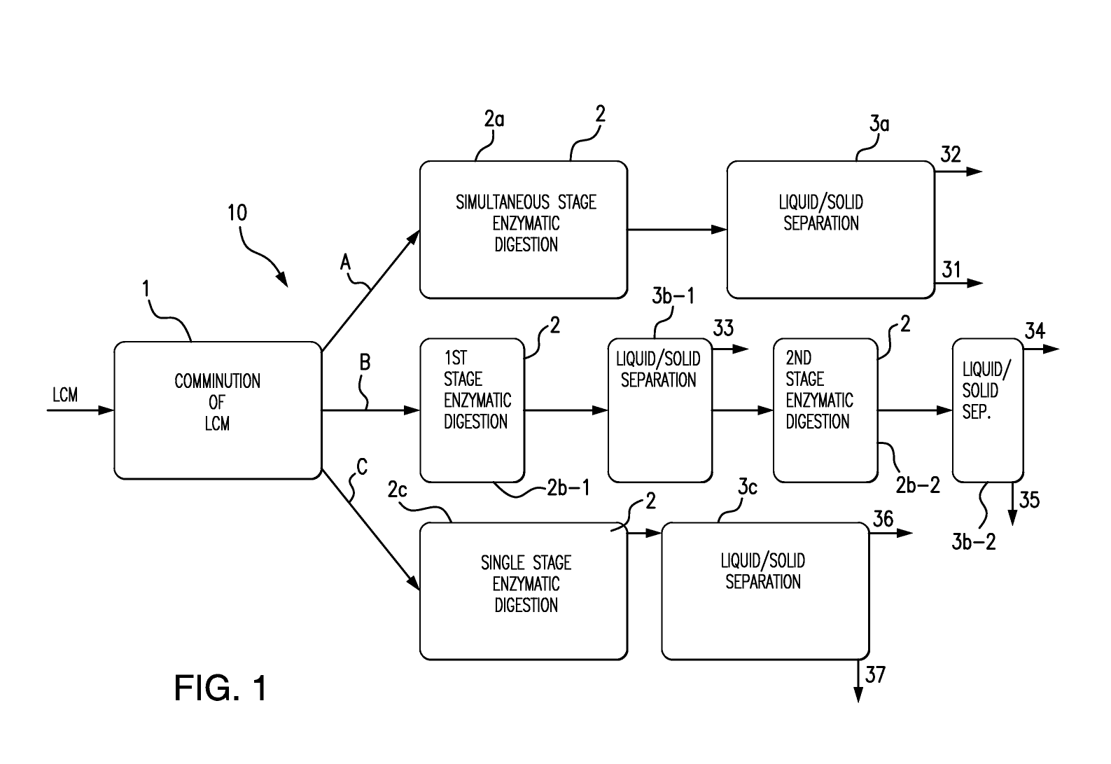

Referring to FIG. 1, a method 10 of the present invention can comprise, for example, a comminution step 1 wherein lignocellulosic material (LCM) is attrited to smaller particle sizes having an increased surface area. The lignocellulosic material can be comminuted to a particle size, for example, of less than about 10 mm, or less than about 7 mm, or less than about 5 mm, or less than about 3 mm, or less than about 2 mm, or less than about 1 mm, or less than about 0.5 mm, or to a size from about 0.001 mm to about 10 mm, or from about 0.01 mm to about 7 mm, or from about 0.1 mm to about 5 mm, or from about 1 mm to about 3 mm, or any other combinations of these ranges values. The particle size can be determined, for example, by mesh sieve series or using a microscope having a micrometer scale for very fine particles. The indicated particles sizes can be absolute or average values. At least 75% (by volume), or at least 90%, or at least 95%, or at least 98%, or at least 99%, or 100%, for example, of the comminuted particles can fall within any of the indicated ranges. The optimal ranges may vary depending on the type of lignocellulosic material to be processed. The size of the lignocellulosic material preferably should not be reduced to a size at which the lignin or cellulose constituents become damaged. While not desiring to be bound to theory, it is thought that the extent and/or rate of enzymatic hydrolytic activity achievable on the comminuted lignocellulosic material in subsequent enzyme digestion may progressively increase with progressively increasing size reduction (increased surface area) up to reaching a threshold where damage to the target constituents or their precursors in the lignocellulosic material occurs.

Then, as indicated in FIG. 1, the comminuted lignocellulosic material can be enzymatically digested and the resulting bioproducts fractionated, for example, according to three different process paths A, B, or C. All three process paths include an enzymatic digestion regimen generally identified as process step(s) 2. For the three different scenarios, the digestion generally includes contacting a mixture comprising the comminuted lignocellulosic material, at least one enzyme blend, and aqueous solution in the absence of any prior non-enzymatic lignin separation treatment of the lignocellulosic material, for enzymatic hydrolysis liberating at least one C6 and/or C5 sugar from an insoluble portion of the lignocellulosic material comprising lignin. As shown in FIG. 1, the digestion regimen 2 can be performed in a single stage or in multiple stages.

In step 2a of process path A, the indicated mixture is simultaneously enzymatically digested with at least one enzyme having cellulytic hydrolytic activity with respect to cellulose, optionally at least one enzyme having hydrolytic activity with respect to hemi-cellulose, and at least one other enzyme having hydrolytic activity with respect to glucan. Then, in step 3a, at least a portion of an aqueous solution containing the sugar is separated from the insoluble portion comprising lignin to provide a lignin-containing product 31 and an aqueous sugar-containing stream 32. The respective lignin and sugar product streams can be separately further processed. For example, the lignin product can be dried before further handling and use or other handling. The sugar product stream can be processed in a biofermentation facility, for example, to make other products, such as ethanol.

In step 2b of process path B, the mixture can be sequentially enzymatically digested in multiple stages 2b-1 and 2b-2. A first enzyme blend comprising at least one enzyme having cellulytic hydrolytic activity with respect to cellulose, and optionally at least one enzyme having hydrolytic activity with respect to hemi-cellulose, can be used in first digestion 2b-1. A second blend of enzymes having hydrolytic activity with respect to glucan can be used in second digestion 2b-2. Liquid/solid separations 3b-1 and 3b-2 are performed after each respective digestion stage 2b-1 and 2b-2 to withdraw an aqueous sugar-containing stream 33 and aqueous residual sugar/wash fluids 34, respectively, which also can be further processed as indicated. A lignin-containing product 35 is produced from the second liquid/solid separation step 3b-2, which also can be further processed as indicated.

In step 2c of process path C, the mixture is enzymatically digested in a single stage with at least one enzyme having cellulytic activity and at least one enzyme having hemi-cellulytic activity in a first digestion. A liquid/solid separations 3c is performed after the single digestion stage 2c to withdraw an aqueous sugar and glucan containing stream 36, and isolate a lignin-containing product 37, which process streams also can be further processed as indicated. Non-hydrolyzed glucan biopolymers, e.g., pectins, that may be present in the aqueous stream 36 of this process path, due to the omission of secondary digestion, may not be acted upon by fermentation yeasts.

In steps 2a, 2b-1, or 2c, the first enzyme blend can comprise combinations of enzymes as described in greater detail where also applicable herein. Depending on the type of lignocellulosic material being processed, degradation of hemicelluloses can simplify the rest of the process and eliminate possible need for potentially complicated downstream separations of hemicellulose and cellulose-derived sugars. Accordingly, a first enzyme blend can combine different enzymes with at least one having hydrolytic activity for cellulose and at least another one having hydrolytic activity for hemi-cellulose. Further, the first enzyme blend used in these steps 2a, 2b-1, and 2c can include, for example, enzymes including at least one endoglucanase or exoglucanase, or both, combined with cellobiase, or beta- or alpha-glucosidase, or combinations thereof. While not desiring to be bound to any theory, it is believed that the presence of the cellobiase (or beta- or alpha-glucosidase), for example, can repress feedback. Feedback refers to a phenomenon where the endoglucanases (or exoglucanase), for example, tend to cleave C2-C5 monomers from the lignocellulosic matrix, which can build-up to a level where they can inhibit the progress of the enzymatic hydrolysis reactions. The cellobiase, or beta- or alpha-glucosidase, for example, can degrade the C2-C5 monomers (e.g., C2-C5 short chain sugars), and thereby repress or prevent feedback with respect to the enzymatic reactions associated with endoglucanase and/or exoglucanase. As an option, cellobiase can be used in this respect to repress feedback. A substantial amount of the C6 and C5 sugars (e.g., at least 50%, or at least 60%, or at least 70%, or at least 80%, or at least 90%, or at least 95%, or at least 99%, percent by weight, based on the original theoretical amount present in the lignocellulosic feedstock) can be liberated from the lignin after the first digestion step 2b-1 in process path B.

The second digestion stage 2b-2 in process path B, which is effectively combined with the first digestion in step 2a of process path A, can use a second blend of enzymes, such as combinations of glucanases and pectinases, etc., or other enzymes described herein for this enzyme blend, that performs a secondary cleaning of the lignin. This secondary cleaning may involve breaking secondary strands of glucans (e.g., pectins), and removing them from the lignin as liberated sugars (e.g., glactoses, mannoses).

The performance of the simultaneous digestion of process path A can be comparable to that of multi-stage digestion of process path B, such as in terms of the lignin purity and amount of liberated C5 and C6 sugars achieved, although not required. For example, the purity of the lignin obtained by process paths A and B can, for example, be within about ±25%, or within about ±20%, or within about ±15%, or within about ±10%, or within about ±5%, or within about ±2%, of each other. The yield of lignin and/or C6 and C5 sugars obtained by process paths A and B can fall within, for example, any of the same tolerance values. A difference in the results of the two process paths A and B can be that the sugars and any smaller molecules liberated from the lignocellulosic matrix by the second blend of enzymes are combined in the product stream of process A with the sugars and other bioproducts liberated by action of the first blend of enzymes, whereas in process path B these bioproducts can be recovered in separate product streams withdrawn from the lignin solids after each digestion stage and associated liquid/solid separation step. In process path C, the secondary cleaning of the lignin with the second blend of enzymes is not included. The lignin products of process path C still can provide high purity lignin products, although they may tend to be less than that achievable with process paths A and B if applied to the same material (depending on the particular lignocellulosic material being processed). Importantly, in process paths A, B, and C, the enzymes used preferably are enzymatically inactive with respect to lignin and preferably leave the lignin intact when liberating the C6 and C5 sugars. Enzymes which can have an enzymatic effect on lignin, such as laccase, preferably are not used, although not categorically excluded.

The present methods can be performed batch-wise or continuously. In either strategy, and irrespective of whether a single or multi-stage enzyme digest sequence such as described herein is used for isolating components of the lignocellulosic material, the lignocellulosic material is comminuted to smaller particle sizes having an increased surface area before at least the initial enzymatic digestion stage. Preferably, neither the original lignocellulosic feedstock nor the comminuted lignocellulosic material receives any non-enzymatic lignin separation treatment before the initial or single enzymatic digestion stage that is applied. If multiple-stages are used, the lignocellulosic material is preferably comminuted to progressively smaller sizes and increased surface area before each successive digestion stage. Further, as indicated, the first and second blends of enzymes can be used simultaneously in a single digestion stage, or separately in separate digestion stages, in the methods of the present invention. As indicated, the first blend of enzymes preferably can include, for example, an enzyme that has cellulytic hydrolytic activity at least with respect to cellulose, and optionally at least one enzyme having hydrolytic activity at least with respect to hemicellulose, or both. The second blend of enzymes preferably can include, for example, an enzyme that has hydrolytic activity at least with respect to glucan polymers, to provide secondary cleaning or “polishing”, for example, of the solid particles containing lignin to remove residual sugars or other non-lignin materials.

The first blend of enzymes can include, for example, at least one enzyme selected from two or more of the following groups 1) to 5) of enzymes:
1) endoglucanase (1,4-beta-D glucanohydrolase), carboxymethylcellulase (CMCase), which can provide attack along the body of the biopolymer chains, for example, and can produce random scisson of cellulose chains yielding glucose and cell-oligosaccharides;
2) exoglucanase (1,4-beta-D glucan cellobiohydrolase), avicelase (C1), which can provide exo (terminal) attack on the non-reducing end of cellulase with cellobiose as the primary structure;
3) cellobiase, beta-glucosidase, alpha-glucosidase, which, as indicated, can repress or prevent feedback with respect to the enzymatic reactions associated with endoglucanase and/or exoglucanase, and can hydrolyze cellobiose to glucose;
4) endo 1,4-beta-xylanase, endo-(1,4)-beta xylanohydrolase, where the xylanases can, for example, degrade hemi-cellulose;
5) beta-1,3-xylanase, 1,3-beta-D-xylosidase, and exo-1,3-beta-xylosidase.
   Any combinations of enzymes selected from multiple groups among groups 1) to 5) can be used in the first enzyme blend. These combinations may include at least one enzyme from at least two of groups 1) to 5), or at least one enzyme from at least three of groups 1) to 5), or at least one enzyme from at least four of groups 1) to 5), or at least one enzyme from at all five of groups 1) to 5). As indicated, one preferred blend is enzymes of groups 1) and/or 2) with enzymes of group 3). Another combination can be enzymes of groups 1), 2), 3), and at least one enzyme of groups 4) and/or 5) or both.

The second blend of enzymes can include, for example, at least one enzyme from two or more of the following groups 1a) to 20a) of enzymes:
1a) endo-1,3(4)-beta-glucanase;
2a) laminarinase (endo-1,3-beta-glucanase);
3a) exo-1,2-1,6-alpha-mannosidase;
4a) beta-D-xylopyranosyl-(1,4)-beta-D-xylopyranose;
5a) alpha-N-arabinofuranosidase;
6a) feruloyl esterase;
7a) endo-1,5-alpha-arabinanase;
8a) pectinase;
9a) polygalacturonase;
10a) pectin esterase;
11a) aspartic protease;
12a) metallo protease;
13a) endo-(1,4)-mannanase;
14a) phytase;
15a) alpha-glucuronidase and beta-glucuronidase;
16a) hexenuronidase;
17a) alkaline phosphatase and acid phosphatase;
18a) alpha-galactosidase and beta-galactosidase;
19a) beta-mannosidase;
20a) alpha-fucosidase.
   Any combinations of these groups of enzymes 1a) to 20a) can be used in the second enzyme blend. These combinations may include, for example, at least one enzyme from at least two of groups 1a) to 20a), or at least one enzyme from at least three of groups 1a) to 20a), or at least one enzyme from at least four of groups 1a) to 20a), or at least one enzyme from at least five of groups 1a) to 20a), and similarly up to a combination of at least one enzyme from each of groups 1a) to 20a). In one method, at least one enzyme from groups 1a), 2a), and 8a), for example, are included in the second enzyme blend. In another method, at least one enzyme from a majority of the groups 1a) to 20a) is used in the second enzyme blend. In another method, at least one enzyme from 2, or 3, or 4, or 5, or 6, or 7, or 8, or 9, or 10, or 11, or 12, or 13, or 14, or 15, or 16, or 17, or 18, or 19, or all 20 of the groups 1a) to 20a) is used in the second enzyme blend.

The total amount of the first blend of enzymes added can be, for example, about 0.001% to about 3% by weight total enzymes, or from about 0.01% to about 1% by weight total enzymes, or from about 0.015% to about 0.8% by weight total enzymes, or from about 0.05% to about 0.6% by weight total enzymes, or from about 0.1% by weight to about 1% by weight total enzymes, or from about 0.2 to about 0.8% by weight total enzymes, or from about 0.25% to about 0.75% by weight total enzymes, or from about 0.4% to about 0.6% by weight total enzymes, or about 0.5% by weight total enzymes, based on lignocellulosic material on a solids weight basis, though other amounts may be used. The proportions of the enzymes used from different groups 1) to 5) can be the same or different. The proportions of enzymes from the first enzyme blend may be, for example, 10-90 wt % total enzymes of groups 1) and 2), 10-90 wt % total enzymes of group 3), and 0-90 wt % total enzymes of groups 4) and 5); or 10-90 wt % total enzymes of groups 1) and 2), 10-90 wt % total enzymes of group 3), and 10-90 wt % total enzymes of group 4) and 5); or 20-40 wt % total enzymes of groups 1) and 2), 20-40 wt % total enzymes of group 3), and 20-40 wt % total enzymes of groups 4) and 5); or other proportions. The total amount of the second blend of enzymes used can be, for example, similar to that indicated for the first enzyme blend, or other effective amounts. The proportions of enzymes in the second blend of enzymes can be, for example, at least 0.5 wt %, or from 0.5% to 50%, or from about 2% to about 10%, of at least one enzyme sourced from a majority of the groups 1a) to 20a), or other indicated numbers of these groups, based on solid weight of the lignocellulosic material or solid fraction derivative thereof to be treated. Any amounts of any enzyme indicated herein can be based on active enzyme.

The enzyme blends can be introduced in solution or in dry form using a substrate. The individual enzymes of each blend can be premixed (e.g., in a common solution), or can be separately introduced individually, or in other lesser included combinations, to the digestor to form the enzyme blend in situ. Enzymes also may be introduced to a digestor in dry form, wherein the enzyme is used with a substrate, such as talc, kaolin, dendrite, and the like. The substrate for dry enzymes may raise additional handling requirements (e.g., to recover the solids attributable to the substrate), and, if used, should be monitored to make sure the substrate does not interfere with the digestion.

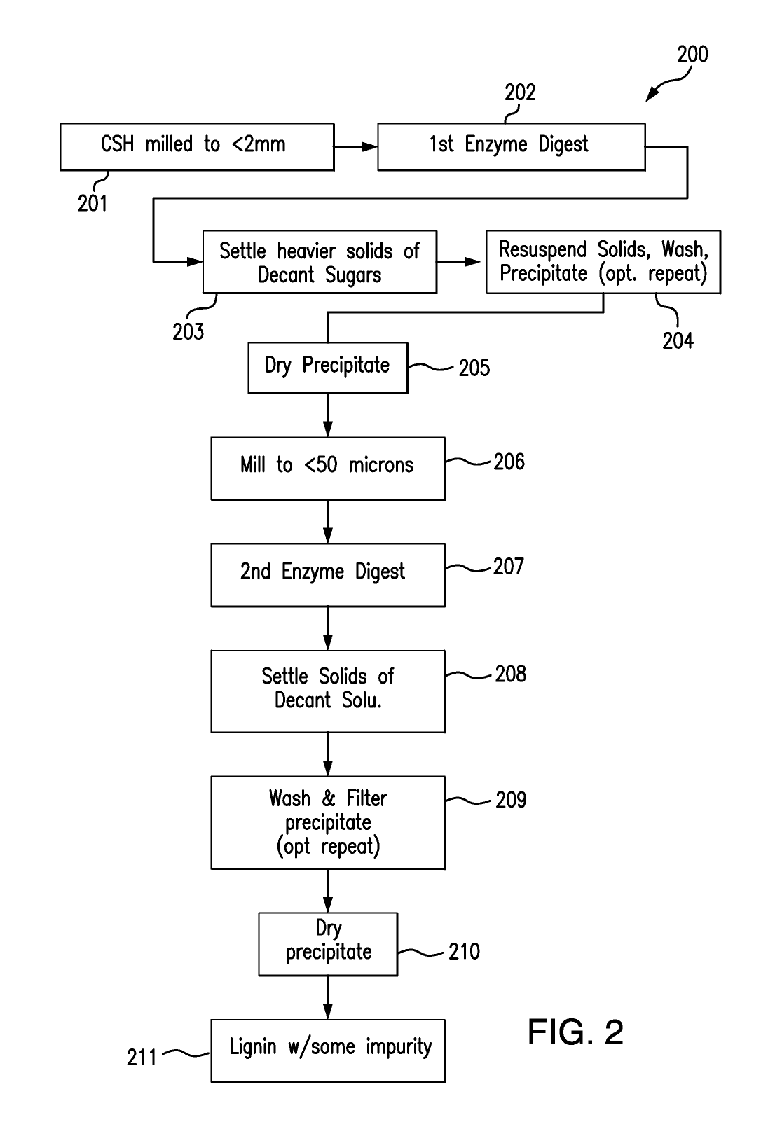

Referring to FIG. 2, the present invention can relate in one more specific application, for example, to a method 200 of enzymatically purifying lignin from herbaceous plants, such as cotton seed hulls, with concomitant liberation of fermentable sugars. In step 201, cotton seed hulls (CSH) are milled to less than 2 mm in size. In step 202, the milled cotton seed hulls receive a first enzymatic digest. In step 203, heavier solids are allowed to settle and sugars in an aqueous phase over the settled solids are decanted. In step 204, the settled solids are resuspended, washed, and recovered for further processing. In step 205, the precipitate is dried. In step 206, the dried precipitate is milled to a size less than 50 μm. In step 207, the milled precipitate receives a second enzymatic digest. In step 208, heavier solids are allowed to settle and residual sugars in an aqueous phase over the settled solids are decanted. In step 209, the settled solids are resuspended, washed, and recovered for further processing. In step 210, the precipitate is dried. In step 211, lignin with some impurity is recovered. This process flow also may be applied to other lignocellulosic materials, with possible adaptations or modifications that can be readily implemented in view of the teachings of the present application.

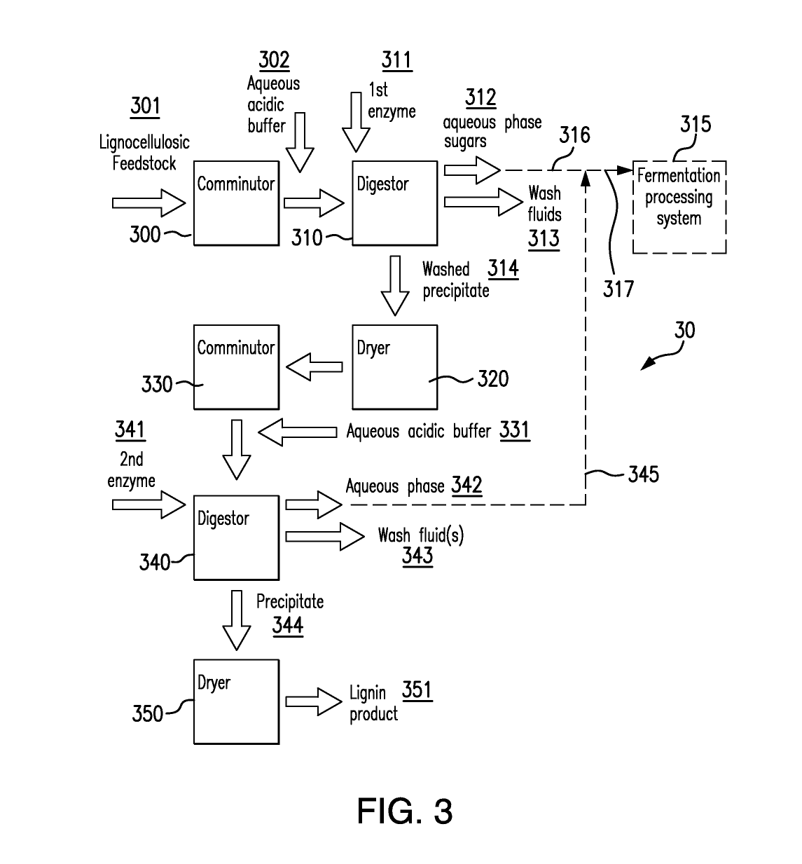

Referring to FIG. 3, method 30 of the present invention can comprise, for example, feeding lignocellulosic feedstock 301, such as cotton seed hulls or other lignocellulosic material, to a first comminutor 300. The feedstock may be plant fiber material that is already chipped, chopped, shredded, and/or ground to some extent. The lignocellulosic feedstock can be attrited at the first comminutor 300 to smaller particle sizes having increased surface area sufficient that enzymatic hydrolysis can be performed on the attrited particles in a more viable manner and without requiring any other preconditioning of the particles. The particle size of the comminuted feedstock provided in this respect, for example, may vary depending on the type of lignocellulosic feedstock material. The comminuted particles size for cotton seed hulls may be, for example, less than about 5 mm, or less than about 3 mm, or less than about 2 mm, or less than about 1.5 mm, or less than about 1 mm, or less than about 1 mm, or less than 0.5 mm, or from about 0.001 to about 5 mm, or from about 0.01 mm to about 3 mm, or from about 0.1 mm to about 2.5 mm, or from about 0.2 mm to about 2 mm, or from about 0.5 mm to about 1.5 mm, or other values. These particles sizes for cotton see hulls can represent absolute or average values. At least 75% (by volume), or at least 90%, or at least 95%, or at least 98%, or at least 99%, or 100%, for example, of the comminuted particles can fall within any of the indicated ranges. These sizes also may apply to other lignocellulosic materials, or suitable comminuted sizes for the subsequent enzyme treatment can be determined empirically using teachings of the present application. The comminution device can be, for example, a dry mill (e.g., a hammer mill, a rotary hammer mill, etc.), wet mill, or other known devices and arrangements for reducing the particle sizes of the feedstock. The comminuted feedstock optionally can be screened, for example, with a mesh sieve screen series, to select size fractions or otherwise further control the particle size distribution before the particulate is further processed in a first enzymatic digestor. The digestor can be any container which has adequate capacity to hold all the mixture components, and which can be equipped with mild temperature control means (e.g., a conventional vessel heating means). The digestor also preferably can be equipped with an agitation device, which can be a conventional agitation device (e.g., impeller, magnetic stirrer, etc.), and the digestor which can be fitted with applicable input inlets and discharge outlets. As can be understood, the types of holding container, agitation device, heating means, and the like, which be more suitable may depend on the scale of the process. The present methods can be implemented, for example, on any scale including industrial, pilot plant, laboratory, or other scales.

A mixture comprising the comminuted lignocellulosic material, a first enzyme blend 311, and a first aqueous solution comprising aqueous acidic buffer 302 is prepared in a first digestor 310. As indicated, the first enzyme blend can be selected, for example, to have cellulytic activity at least with respect to cellulose, optionally hemicellulose, or both. As indicated, enzymes that may modify the lignin, such as laccase, are less preferred where purity of the lignin product is desired. The enzyme and buffer can be introduced at the digestor or combined with the comminuted lignocellulosic feedstock before it is introduced into the digestor. The first aqueous solution can comprise, for example, an aqueous acidic organic buffer solution such as a citrate buffer. The acidic buffer solution can have pH, for example, of about 4.5 to about 6.9, or from about 4.7 to about 5.5, or from about 4.8 to about 5.2. The acid buffered solution is not acidic enough to cause acid hydrolysis of the lignocellulosic material or impair the enzymatic hydrolysis mechanisms. The mixture of feedstock, enzyme, buffer, and solution is agitated to provide and maintain a more uniform combination during digestion. The mixture can be agitated under mild heating, for example, for a sufficient duration for liberating C6 and/or C5 sugars from a first insoluble portion of the lignocellulosic material comprising lignin. The mixture can be digested, for example, for at least about 1 hour at a temperature of at least about 30° C. The digestion temperature need not exceed, for example, about 60° C. and lower temperatures can be practically used. The digestion also can be conducted at approximately atmospheric pressure without a need to pressurize the contents of the digestor to achieve the desired enzymatic activity on the lignocellulosic particles. The mixture can be digested, for example, for at least about 3 hours, or for at least about 6 hours, or for at least about 9 hours, or for at least about 12 hours, or for at least about 15 hours, or for at least about 18 hours, or for at least about 21 hours, or for at least 24 hours, at a temperature of at least about 35° C., or at least about 40° C. or at least about 45° C., or at least about 50° C., or from about 30° C. to about 50° C., or other temperatures. A portion of the first aqueous solution 312 containing the at least one C6 and/or C5 sugar is decanted from the first insoluble portion. The C6 and/or C5 sugars can comprise at least one of solubilized C6 and/or C5 sugars, suspended C6 and/or C5 sugar particulate, or both. The precipitated lignin-containing material in the vessel can be separated from aqueous phase containing the C6 and/or C5 sugars by decanting or siphoning off the aqueous phase of the enzymatically digested mixture. Filtration (e.g., screen filtration), centrifugation, and other known means of separating solids from aqueous phases may be used. Decanting or siphoning, or other separation methods which withdraw a liquid phase while the settled solid phase is left relatively undisturbed, are preferred separation methods for isolating the lignin-containing solid fractions of the digested mixtures. Other techniques, such as centrifugation, may lead to increased non-lignin impurities in the solid fraction. The decanted sugars may be transmitted to a fermentation processing system 315. Techniques are known which can be used for fermenting these sugars, which may generally include steps of microbial fermentation of the sugar solution, distillation to produce roughly 95% pure alcohol, and dehydration by molecular sieves to bring the ethanol concentration to over 99.5%, or other known fermentation methods. The formation of ethanol from the sugars can be accomplished, for example, by yeasts such as Saccharomyces cerevisiae, such as described in U.S. Pat. No. 2,802,774, and Fusarium oxysporum. Other useful microorganisms are the ethanol-producing bacillus described, for example, in U.S. Pat. No. 4,094,742, which are all incorporated herein in their entireties by reference.

The insoluble portion in the first digestor can be resuspended and washed at least once. An initial wash used may be mildly acidic, and any subsequent wash fluids can be water alone. The settled solids can be washed in the same vessel as where digestion is performed, or, alternatively, a separate vessel can be used for washing the settled solids. The wash fluids 313 can be withdrawn or removed by any convenient solid/liquid separation technique (e.g., decanting, siphoning, centrifuging, screen filtering, etc.). The washed precipitate 314 can be dried in a dryer 320. The dried insoluble portion can be comminuted to smaller particles sizes having a second increased surface area at a comminutor 330. This comminutor can be the same or different that the previous unit. The dried particles can be comminuted at comminutor 330 to even smaller sizes than provided to the previous digestor to make additional surface area available for enzymatic action in a subsequent digestion. The comminuted particles size produced for cotton seed hulls at the second comminutor unit 330 may be, for example, less than about 50 μm, or less than about 40 μm, or less than about 30 μm, or less than about 20 μm, or less than 10 μm, or from about 1 μm to about 50 μm, or from about 5 μm to about 40 μm, or from about 7.5 μm to about 30 μm, or other values. These particles sizes can represent absolute or average values. At least 75% (by volume), or at least 90%, or at least 95%, or at least 98%, or at least 99%, or 100%, for example, of the comminuted cotton seed hull particles can fall within any of the indicated ranges. These particles sizes also may apply to other lignocellulosic materials, or suitable comminuted sizes for the subsequent enzyme treatment can be determined empirically using teachings of the present application.

A second mixture comprising the comminuted first insoluble portion, a second enzyme blend 341, and a second aqueous solution comprising an aqueous acidic buffer 331, which can be similar to that used in the first digestor, are fed to a second digestor 340. The second enzyme blend can be introduced in solution or dry. As indicated, the second enzyme blend can be selected, for example, to have cellulytic hydrolytic activity at least with respect to glucans. The second mixture can be agitated under mild heating for a sufficient duration for liberating residual C6 and/or C5 sugars from a second insoluble portion comprising lignin. The temperature and pressure conditions indicated for the first digestor 310 can be, for example, used in the second digestor 340. As with the first digestor, the digestion temperature need not exceed, for example, about 60° C. and lower temperatures and atmospheric pressures can be practically used. An aqueous phase containing residual sugars 342 can be decanted from the second digestor, and optionally transmitted to the fermentation processing system 315 or other processing systems. The second insoluble portion comprising lignin can be resuspended and washed. The remaining precipitate 344 can be fed to a dryer 350 and the lignin product 351 collected. Although a two stage comminution and digestion process is illustrated, it will be understand that a single stage process may be used, or a three stage process, or a four stage process, or even a higher-stage process, also may be used.

All process vessels and equipment illustrated in FIG. 3 can be fitted with suitable inlets for introduction of all applicable feeds and discharge outlets for removal of materials as indicated, such as using conventional fittings and equipment for such purposes. Fluid materials can be gravity drained or pumped from process vessel to vessel. The lignocellulosic materials which can be processed using this arrangement are not necessarily limited, and include the previously indicated materials herein. One skilled in the art will recognize that other lignocellulose-containing feedstocks exist and can be fractionated by practicing the methods of the present invention. Further, although not shown in FIG. 3, enzyme recovery can be implemented on aqueous sugar stream 316, or on aqueous stream 345, or a combined stream 317 of them. Enzyme content from these aqueous streams may be separated, for example, by suitable separation processes, such as using reverse osmosis, microfiltration, or other methods, and recycled for re-use in a digestor in the above-indicated system, where applicable. Fresh enzymes can be used exclusively in the present methods, or recycled enzymes can be used exclusively, or combinations of fresh and recycled enzymes can be used in any proportions in the present methods.

By practicing the methods of the present invention, lignocellulosic material can be fractionated into at least lignin and C6 and/or C5 sugars (e.g., fermentable sugars). Other materials also may be present in the indicated one or more of the product streams of the methods. The product yields obtained by methods of the present invention can be high. For purposes herein, “yield” is the mass of a certain product recovered, divided by the theoretical maximum based on the amount present in the initial lignocellulosic material (accounting for water addition for the enzymatic hydrolysis reactions). The yield of lignin can be, for example, at least about 50%, or at least about 60%, or at least about 70%, or at least about 80%, or at least about 90%, or at least about 91%, or at least about 92%, or at least about 93%, or at least about 94%, or at least about 95%, or at least about 96%, or at least about 97%, or at least about 98%, or at least about 99%, or more. The yield of C6 and C5 sugars can be, for example, at least about 50%, or at least about 60%, or at least about 70%, or at least about 80%, or at least about 85%, or at least about 90%, or at least about 95%, or at least about 96%, or at least about 97%, or at least about 98%, or at least about 99%, or more.

The lignin that is obtained can be a high-quality, relatively pure, low-molecular-weight lignin that does not contain sulfur. The lignin products of the methods of the present invention have many possible applications. Lignin can be burned for energy production. Some other potential applications for lignin include carbon-fiber production, asphalt production, and as a component in biopolymers. These uses include, for example, oil well drilling additives, concrete additives, dyestuffs dispersants, agriculture chemicals, animal feeds, industrial binders, specialty polymers for paper industry, precious metal recovery aids, wood preservation, sulfur-free lignin products, automotive brakes, wood panel products, biodispersants, polyurethane foams, epoxy resins, printed circuit boards, emulsifiers, sequestrants, water treatment formulations, strength additive for wallboard, adhesives, raw materials for vanillin, xylitol, and as a source for paracoumaryl, coniferyl, sinapyl alcohol.

The C6 and C5 sugar products of the present process can be fermented, for example, into ethanol, n-butanol, acetone, organic acids, baker's yeast, or any other product of cellular metabolism of the chosen microorganism for fermentation. Other commercial products that can be manufactured from sugars include, for example, feed additives for animals, xylitol sweetener, furfural, and other useful products.

The methods of the present invention can avoid the practice of some prior processes of using concentrated acids or alkalis to liberate components of lignocellulosic materials. The enzymatic treatments of the present methods do not rely upon these harsh chemical treatments for lignin component release and can eliminate the production of lignosulfonates associated with conventional pulp treatments which have been used in papermaking. The enzymatic process of the present invention can be significantly less energy intensive than conventional thermochemical processes using high pressure steam, which also can detract from the value of the pure lignin components. Further, enzymatic treatments used in methods of the present invention do not affect or at least do not significantly affect down stream processing unlike the use of some chelating agents such as EDTA. Accordingly, the need for post processing subsystems to assist final purification steps for the lignin is reduced or avoided altogether using the methods of the present invention. Unlike irradiation treatments, enzymatic approaches used in methods of the present invention do not alter the lignin components, but gradually release them intact, and have reduced costs as no irradiation equipment is needed. Impure recalcitrant materials are digested. Natural enzymes can be used in methods of the present invention for the release of components without need of recombinant polypeptides to facilitate any final purification of the lignin components. Live anaerobic live microorganisms are not necessary when using only the enzyme solutions in the absence of the organisms. There is no need to employ extreme measures to reduce impurities which may be recalcitrant to the feedstock solution due to natural removal of these impurities by mild enzymatic hydrolysis which assists conversion of liberated natural lignin components and lignin derivatives. Further, the methods of the present invention lend themselves to recycling of the enzymatic components which possibly lowers the cost of the recovery of the valuable discrete materials. The present methods can be environmentally friendly and sustainable from a cost of operation and community perspective.

The present invention includes the following aspects/embodiments/features in any order and/or in any combination:
1. The present invention relates to a method for isolating components of lignocellulosic material comprising:
   comminuting lignocellulosic material to form comminuted lignocellulosic material;
   enzymatically digesting a mixture comprising the comminuted lignocellulosic material, at least one blend of enzymes, and aqueous solution in the absence of any prior non-enzymatic lignin separation from the lignocellulosic material, for enzymatic hydrolysis liberating at least one C6 and/or C5 sugar from an insoluble portion of the lignocellulosic material comprising lignin; and
   separating at least a portion of the aqueous solution containing the at least one C6 and/or C5 sugar from the insoluble portion comprising lignin.
2. The method of any preceding or following embodiment/feature/aspect, wherein the aqueous solution comprises an aqueous acidic organic buffer solution.
3. The method of any preceding or following embodiment/feature/aspect, wherein the at least one C6 and/or C5 sugar in the aqueous solution comprises solubilized C6 and/or C5 sugar, or suspended C6 and/or C5 sugar particulate, or any combinations thereof.
4. The method of any preceding or following embodiment/feature/aspect, wherein the enzymatically digesting of the comminuted lignocellulosic material in the aqueous solution with the enzyme blend comprises agitating the mixture for at least about 1 hour at a temperature of from about 30° C. to about 60° C., and the separating comprises discontinuing the agitating and decanting the at least a portion of the aqueous solution containing the at least one C6 and/or C5 sugar from the insoluble portion comprising settled solids.
5. The method of any preceding or following embodiment/feature/aspect, wherein the lignocellulosic material is cotton seed hulls, grain hulls, sugarcane bagasse, corn stover, corn cobs, straw, switchgrass, leaves, stalks, plant shells, softwood pieces, hardwood pieces, sawdust, papermill pulp, waste paper, recycled paper, or any combinations thereof.
6. The method of any preceding or following embodiment/feature/aspect, wherein the lignocellulosic material comprises cotton seed hulls.
7. The method of any preceding or following embodiment/feature/aspect, wherein the at least one enzyme blend comprises a first enzyme blend comprising at least one enzyme from at least two of groups 1) to 5):
   1) endoglucanase, carboxymethylcellulase;
   2) exoglucanase, avicelase;
   3) cellobiase, beta-glucosidase, alpha-glucosidase;
   4) endo 1,4-beta-xylanase, endo-(1,4)-beta xylanohydrolase;
   5) beta-1,3-xylanase, 1,3-beta-D-xylosidase, and exo-1,3-beta-xylosidase.
8. The method of any preceding or following embodiment/feature/aspect, wherein first enzyme blend comprises:
   at least one enzyme from groups 1) and/or 2),
   at least one enzyme of group 3), and
   at least one enzyme of groups 4) and/or 5).
9. The method of any preceding or following embodiment/feature/aspect, wherein the at least one enzyme blend further comprises a second enzyme blend comprising at least one enzyme from at least two of groups 1a) to 20a):
   1a) endo-1,3(4)-beta-glucanase;
   2a) laminarinase (endo-1,3-beta-glucanase);
   3a) exo-1,2-1,6-alpha-mannosidase;
   4a) beta-D-xylopyranosyl-(1,4)-beta-D-xylopyranose;
   5a) alpha-N-arabinofuranosidase;
   6a) feruloyl esterase;
   7a) endo-1,5-alpha-arabinanase;
   8a) pectinase;
   9a) polygalacturonase;
   10a) pectin esterase;
   11a) aspartic protease;
   12a) metallo protease;
   13a) endo-(1,4)-mannanase;
   14a) phytase;
   15a) alpha-glucuronidase and beta-glucuronidase;
   16a) hexenuronidase;
   17a) alkaline phosphatase and acid phosphatase;
   18a) alpha-galactosidase and beta-galactosidase;
   19a) beta-mannosidase;
   20a) alpha-fucosidase.
10. The method of any preceding or following embodiment/feature/aspect, wherein yield of lignin is at least about 90%.
11. The method of any preceding or following embodiment/feature/aspect, wherein yield of C6 and/or C5 sugars is at least about 90%.
12. A method for isolating components of lignocellulosic material comprising:
   a) comminuting lignocellulosic material to a comminuted lignocellulosic material;
   b) enzymatically digesting a first mixture comprising the comminuted lignocellulosic material, a first enzyme blend, and a first aqueous solution in the absence of any prior non-enzymatic lignin separation treatment of the lignocellulosic material, for a first enzymatic hydrolysis liberating at least one C6 and/or C5 sugar from a first insoluble portion of the lignocellulosic material comprising lignin;
   c) separating at least a portion of the first aqueous solution containing the at least one C6 and/or C5 sugar from the first insoluble portion;
   d) comminuting the first insoluble portion to a comminuted first insoluble portion;
   e) enzymatically digesting a second mixture comprising the comminuted first insoluble portion, a second enzyme blend, and a second aqueous solution, for a second enzymatic hydrolysis liberating at least one C6 and/or C5 sugar from a second insoluble portion comprising lignin; and
   f) separating the second insoluble portion comprising lignin from the second aqueous solution comprising the at least one C6 and/or C5 sugar.
13. The method of any preceding or following embodiment/feature/aspect, wherein the comminuting of the lignocellulosic material comprises milling the lignocellulosic material to a size less than about 2 mm.
14. The method of any preceding or following embodiment/feature/aspect, wherein the comminuting of the first insoluble portion comprises milling or grinding the first insoluble portion to a size less than about 50 microns.
15. The method of any preceding or following embodiment/feature/aspect, further comprising, after the separating, washing the first insoluble portion at least once and drying the washed first insoluble portion before the comminuting of the first insoluble portion.
16. The method of any preceding or following embodiment/feature/aspect, wherein the first aqueous solution comprises an aqueous acidic organic buffer solution.
17. The method of any preceding or following embodiment/feature/aspect, wherein the at least one C6 and/or C5 sugar in the first aqueous solution comprises solubilized C6 and/or C5 sugar, or suspended C6 and/or C5 sugar particulate, or any combinations thereof.
18. The method of any preceding or following embodiment/feature/aspect, wherein the enzymatically digesting of the comminuted lignocellulosic material in the first aqueous solution with the first enzyme blend comprises agitating the first mixture for at least about 1 hour at a temperature of from about 30° C. to about 60° C., and the separating comprises discontinuing the agitating and decanting the at least a portion of the first aqueous solution containing the at least one C6 and/or C5 sugar from the first insoluble portion comprising settled solids.
19. The method of any preceding or following embodiment/feature/aspect, wherein the lignocellulosic material is cotton seed hulls, grain hulls, sugarcane bagasse, corn stover, corn cobs, straw, switchgrass, leaves, stalks, plant shells, softwood pieces, hardwood pieces, sawdust, papermill pulp, waste paper, recycled paper, or any combinations thereof.
20. The method of any preceding or following embodiment/feature/aspect, wherein the lignocellulosic material comprises cotton seed hulls.
21. The method of any preceding or following embodiment/feature/aspect, wherein the first enzyme blend comprises at least one enzyme from at least two of groups 1) to 5):
   1) endoglucanase, carboxymethylcellulase;
   2) exoglucanase, avicelase;
   3) cellobiase, beta-glucosidase, alpha-glucosidase;
   4) endo 1,4-beta-xylanase, endo-(1,4)-beta xylanohydrolase;
   5) beta-1,3-xylanase, 1,3-beta-D-xylosidase, and exo-1,3-beta-xylosidase.
22. The method of any preceding or following embodiment/feature/aspect, wherein the second enzyme blend comprises at least one enzyme from at least two of groups 1a) to 20a):
   1a) endo-1,3(4)-beta-glucanase;
   2a) laminarinase (endo-1,3-beta-glucanase);
   3a) exo-1,2-1,6-alpha-mannosidase;
   4a) beta-D-xylopyranosyl-(1,4)-beta-D-xylopyranose;
   5a) alpha-N-arabinofuranosidase;
   6a) feruloyl esterase;
   7a) endo-1,5-alpha-arabinanase;
   8a) pectinase;
   9a) polygalacturonase;
   10a) pectin esterase;
   11a) aspartic protease;
   12a) metallo protease;
   13a) endo-(1,4)-mannanase;
   14a) phytase;
   15a) alpha-glucuronidase and beta-glucuronidase;
   16a) hexenuronidase;
   17a) alkaline phosphatase and acid phosphatase;
   18a) alpha-galactosidase and beta-galactosidase;
   19a) beta-mannosidase;
   20a) alpha-fucosidase.
23. The method of any preceding or following embodiment/feature/aspect, wherein yield of lignin is at least about 90%.
24. The method of any preceding or following embodiment/feature/aspect, wherein yield of the at least one C6 and/or C5 sugar is at least about 50% based on the original amount present in the lignocellulosic material.
25. The method of any preceding or following embodiment/feature/aspect, further comprising biofermentation processing of the at least one C6 and/or C5 sugar.
26. A system for isolating components of lignocellulosic material comprising:
   at least one comminutor for comminuting lignocellulosic material;
   at least one digestor comprising a vessel equipped with an internal agitator, wherein the vessel is operable for holding a mixture of the comminuted lignocellulosic material, at least one enzyme blend, and aqueous solution in the absence of any prior non-enzymatic lignin separation treatment of the lignocellulosic material, and operable for enzymatically digesting the mixture with agitation for enzymatic hydrolysis liberating at least one C6 and/or C5 sugar from an insoluble portion of the lignocellulosic material comprising lignin; and
   a separator for separating at least a portion of the aqueous solution containing the at least one sugar from the insoluble portion comprising lignin.
27. The system of any preceding or following embodiment/feature/aspect, further comprising:
   a dryer for drying the insoluble portion comprising lignin;
   a comminutor for comminuting the first insoluble material;
   a digestor comprising a vessel equipped with an internal agitator, wherein the vessel is operable for holding a mixture of the comminuted first insoluble material, at least one enzyme, and aqueous solution, and contacting the mixture with agitation for enzymatic hydrolysis liberating at least one C6 and/or C5 sugar from an insoluble portion of the lignocellulosic material comprising lignin; and
   a separator for separating at least a portion of the aqueous solution containing the at least one sugar from the insoluble portion comprising lignin.
28. The system of any preceding or following embodiment/feature/aspect, further comprising a biofermentation processing system adapted to receive the liberated C6 and/or C5 sugar from the digestor and ferment the C6 and/or C5 sugar into ethanol.

The present invention can include any combination of these various features or embodiments above and/or below as set forth in sentences and/or paragraphs. Any combination of disclosed features herein is considered part of the present invention and no limitation is intended with respect to combinable features.

The following examples are intended to illustrate, not limit, the present invention. In the following examples, all parts are proportions by weight unless otherwise specified.

### EXAMPLES

### Example 1

Experiments were conducted to evaluate the effectiveness of enzymes on comminuted cotton seed hulls as a source of lignocellulosic material for lignin purification and sugar isolation.

### Experimental Procedures

### Purification Method

Cotton seed hulls were obtained from Buckeye Technologies (Memphis, Tenn.). The cotton seed hulls were dry milled to sizes less than 2 mm. Milling was performed by hammer milling or comparable milling. The sizes of the milled hulls were determined by mesh sieve. Approximately 220 grams of the milled cotton seed hulls was made up as a 10% solids solution in 50 mM citrate buffer pH 5.0. The 10% solids solution of cotton seed hulls contained 220 grams cotton seed hulls in approximately 2200 ml solution.

The 50 mM citrate buffer (pH 5.0) was prepared in the following manner. A pH 5.0 citrate buffer stock was prepared by mixing 210 grams citric acid monohydrate in 900 grams water, then gradually adding 100 ml 50% NaOH with stirring, which yielded 1 liter of pH 5.0 citrate buffer stock. The pH 5.0, 50 mM (0.05 M) citrate buffer was prepared using the buffer stock by diluting 50 to 55 ml of the pH 5.0 citrate buffer stock solution to 1 liter water to yield 50 mM citrate buffer for use as the buffer solution for the cotton seed hull solids. A pH meter was used to monitor the pH of the citrate buffer, which was adjusted as needed to provide and maintain a pH of 5.0 by adding 50% NaOH or concentrated HCl dropwise as necessary.

The temperature of the 10% cotton seed hull solution was raised to approximately 50° C. and held at that temperature. The heated solution was amended with 0.5% first enzyme solution in a 4 Liter fermentation beaker as a digestor. The first enzyme solution contained a mixture of enzymes comprised of ⅓ part by weight Cellic™ CTEC, an enzyme preparation including endogluconases, cellobiohydrolases, and beta-glucosidases, obtained from Novozymes North America (Franklinton, N.C.), ⅓ part by weight glucosidases obtained from Sigma-Aldrich (St. Louis, Mo.), and ⅓ part by weight H-TEC xylanase obtained from Novozymes North America. The resulting mixture was agitated using a magnetic stirrer at approximately 200 rpm for 24 hours. After 24 hours, the agitation was discontinued and solids were allowed to settle. The aqueous phase above the settled solids was decanted from the digestor. The decanted aqueous phase contains sugars and suspended fine sugar particulates. Larger, heavier solids only were retained as settled precipitate in the digestor. The settled solids were resuspended in the digestor by addition of water and acidified with HCl to a pH 2.5, and then agitated to provide a mild acid wash step. Agitation was discontinued after washing the solids, and the solids were allowed to settle again. The aqueous phase of wash fluid was decanted from the digestor leaving only solids. The solids were resuspended in water only (not necessary to acidify), agitated, and the solids were recollected after discontinuing agitation. This wash procedure can be repeated until the aqueous phase is clear or almost clear. The solids were then dried in a hot air oven at a temperature of approximately 50° C. overnight. The dried precipitate (solids) are milled to less than 50 microns in size. A mortar and pestle were used to grind the solids to a very fine powder. A microscope with a micrometer scale was used to measure the size of the ground solids. Periods of grinding the solids were interrupted to check the size of the ground solids until the indicated particle size was obtained and then grinding was discontinued. This powder is not particularly hydroscopic. The milled powder is again made up in citrate buffer pH 5.0, 0.05 mM to a 10% solids solution as before. The solution was agitated with a magnetic stirrer at approximately 200 rpm and amended with a second enzyme solution. The second enzyme solution was an enzyme blend in solution comprising ⅓ part by weight CELLUSUB obtained from Specialty Enzymes and Biotechnologies (Chino, Calif.), and ⅔ part by weight penicillin sp. ferment, which was a preparation made on-site which included all or essentially all the enzymes of the indicated second blend of enzymes. The resulting mixture was digested at a temperature of approximately 45° C. for 6 hours. Agitation was discontinued and, as before, solids (precipitate) were allowed to settle. The aqueous phase above the settled solids was decanted, removing as much fine particulate suspended in the aqueous phase as possible while leaving the heavier settled solids in the digestor. The settled solids can be washed with non-acidified water to remove more fines, until the aqueous phase is clear or almost clear. The precipitate is dried in the hot air oven under similar conditions as the indicated first drying procedure. The finished dried material is lignin with some small impurity.

### Lignin Assay

An acetyl bromide assay solution was used to identify and quantify the lignin in the cotton seed hulls and finished lignin product. The acetyl bromide assay for lignin followed published procedures of Ilyama, K., et al., “Determination of Lignin in Herbaceous Plants by an Improved Acetyl Bromide Procedure”, J Sci Food Agric 1990; and Hatfield, R. D., et al., “Using the Acetyl Bromide Assay To Determine Lignin Concentrations in Herbaceous Plants: Some Cautionary Notes”, J Agric Food Chem 1999. The assay method generally comprised the following procedure.

(1) To each test tube 10-15 mg of dry ground sample is added. Samples are ground to a powder, but will work up to a maximum of a 20 mesh screen. Also, sample weights may range from 2 to 40 mg and still give good results, but using at least 10 mg ensures more accurate mass, and using no greater than 15 mg ensures complete digestion. One tube is always used as a procedural blank and at least two tubes for “standards.”

(2) To each tube add 10 ml DI H₂O. Tubes are placed in dry block at 65° C. (set at 85) and heated for one hour, with stirring every 10 minutes.

(3) The sample is filtered through GF/A glass fiber filter and rinse three times with each of the following solutions in this order: water>ethanol>acetone>diethyl ether. A couple of minutes is allowed for each rinse.

(4) The filter disk is placed in glass 20 ml scintillation vial (without lids) and heated overnight at 70° C.

(5) 2.5 ml of 25% AcBr in acetic acid is added to each vial.

(6) The vials are placed in a 50° C. oven for 2 hours with lids, and swirled occasionally.

(7) The samples are cooled in a refrigerator. Volumetric flasks are prepared with 10 ml of 2N sodium hydroxide, and 12 ml of acetic acid. Each sample is transferred from the scintillation vials to volumetric flasks. The filter paper well is rinsed well with acetic acid into the volumetric and bring to volume with acetic acid.

(8) The samples are allowed to settle for at last an hour, and preferably overnight, and then measuring them with absorbance measured at 280 nm.

### Assay Results

The assay results showed that the 220 grams of cotton seed hulls assayed at approximately 20% pure lignin content. The finished lignin product assayed at about 95% pure lignin content.

The results of the experimental studies indicate that the vast amount of sugars were removed after the first enzyme digest. After the second digest, mostly lignin remains with some impurity. Fraction inventory demonstrated that approximately 60 grams of the 220 grams original remained as solids after 1st digest. Therefore, the vast amount of sugars were removed initially in the first digest. The refining second digest only performs a secondary cleaning of the lignin. While not desiring to be bound to theory, the fine milling (powdering) of the solids before the enzyme treatment is thought to increase surface and greatly assists the removal of residual sugars upon the second enzyme digest (concentrated carbohydrases) used for the refining of the solids. Accordingly, protocols or processes preferably can work with the smallest particle size possible creating the largest surface area which allows the enzymes to access the available sugars. It may be possible to perform a primary digest at 20% to 30% solids, based on total weight of the mixture to be digested. It also follows that the amount of solids after the first digest could be collected for a period of time and then milled to a refining size (e.g., less than about 50 microns), before performing the second digest stage.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A method for isolating components of lignocellulosic material comprising:
comminuting lignocellulosic material to form comminuted lignocellulosic material;
enzymatically digesting a mixture comprising the comminuted lignocellulosic material, at least one blend of enzymes, and aqueous solution in the absence of any prior non-enzymatic lignin separation from the lignocellulosic material, for enzymatic hydrolysis liberating at least one C6 and/or C5 sugar from an insoluble portion of the lignocellulosic material comprising lignin; and
separating at least a portion of the aqueous solution containing the at least one C6 and/or C5 sugar from the insoluble portion comprising lignin.

2. The method of claim 1, wherein the aqueous solution comprises an aqueous acidic organic buffer solution.

3. The method of claim 1, wherein the at least one C6 and/or C5 sugar in the aqueous solution comprises solubilized C6 and/or C5 sugar, or suspended C6 and/or C5 sugar particulate, or any combinations thereof.

4. The method of claim 1, wherein the enzymatically digesting of the comminuted lignocellulosic material in the aqueous solution with the enzyme blend comprises agitating the mixture for at least about 1 hour at a temperature of from about 30° C. to about 60° C., and the separating comprises discontinuing the agitating and decanting the at least a portion of the aqueous solution containing the at least one C6 and/or C5 sugar from the insoluble portion comprising settled solids.

5. The method of claim 1, wherein the lignocellulosic material is cotton seed hulls, grain hulls, sugarcane bagasse, corn stover, corn cobs, straw, switchgrass, leaves, stalks, plant shells, softwood pieces, hardwood pieces, sawdust, papermill pulp, waste paper, recycled paper, or any combinations thereof.

6. The method of claim 1, wherein the lignocellulosic material comprises cotton seed hulls.

7. The method of claim 1, wherein the at least one enzyme blend comprises a first enzyme blend comprising at least one enzyme from at least two of groups 1) to 5):
1) endoglucanase, carboxymethylcellulase;
2) exoglucanase, avicelase;
3) cellobiase, beta-glucosidase, alpha-glucosidase;
4) endo 1,4-beta-xylanase, endo-(1,4)-beta xylanohydrolase;
5) beta-1,3-xylanase, 1,3-beta-D-xylosidase, and exo-1,3-beta-xylosidase.

8. The method of claim 7, wherein first enzyme blend comprises:
at least one enzyme from groups 1) and/or 2),
at least one enzyme of group 3), and
at least one enzyme of groups 4) and/or 5).

9. The method of claim 1, wherein the at least one enzyme blend further comprises a second enzyme blend comprising at least one enzyme from at least two of groups 1a) to 20a):
1a) endo-1,3(4)-beta-glucanase;
2a) laminarinase (endo-1,3-beta-glucanase);
3a) exo-1,2-1,6-alpha-mannosidase;
4a) beta-D-xylopyranosyl-(1,4)-beta-D-xylopyranose;
5a) alpha-N-arabinofuranosidase;
6a) feruloyl esterase;
7a) endo-1,5-alpha-arabinanase;
8a) pectinase;
9a) polygalacturonase;
10a) pectin esterase;
11a) aspartic protease;
12a) metallo protease;
13a) endo-(1,4)-mannanase;
14a) phytase;
15a) alpha-glucuronidase and beta-glucuronidase;
16a) hexenuronidase;
17a) alkaline phosphatase and acid phosphatase;
18a) alpha-galactosidase and beta-galactosidase;
19a) beta-mannosidase;
20a) alpha-fucosidase.

10. The method of claim 1, wherein yield of lignin is at least about 90%.

11. The method of claim 1, wherein yield of C6 and/or C5 sugars is at least about 90%.

12. A method for isolating components of lignocellulosic material comprising:
a) comminuting lignocellulosic material to a comminuted lignocellulosic material;
b) enzymatically digesting a first mixture comprising the comminuted lignocellulosic material, a first enzyme blend, and a first aqueous solution in the absence of any prior non-enzymatic lignin separation treatment of the lignocellulosic material, for a first enzymatic hydrolysis liberating at least one C6 and/or C5 sugar from a first insoluble portion of the lignocellulosic material comprising lignin;
c) separating at least a portion of the first aqueous solution containing the at least one C6 and/or C5 sugar from the first insoluble portion;
d) comminuting the first insoluble portion to a comminuted first insoluble portion;
e) enzymatically digesting a second mixture comprising the comminuted first insoluble portion, a second enzyme blend, and a second aqueous solution, for a second enzymatic hydrolysis liberating at least one C6 and/or C5 sugar from a second insoluble portion comprising lignin; and
f) separating the second insoluble portion comprising lignin from the second aqueous solution comprising the at least one C6 and/or C5 sugar.

13. The method of claim 12, wherein the comminuting of the lignocellulosic material comprises milling the lignocellulosic material to a size less than about 2 mm.

14. The method of claim 12, wherein the comminuting of the first insoluble portion comprises milling or grinding the first insoluble portion to a size less than about 50 microns.

15. The method of claim 12, further comprising, after the separating, washing the first insoluble portion at least once and drying the washed first insoluble portion before the comminuting of the first insoluble portion.

16. The method of claim 12, wherein the first aqueous solution comprises an aqueous acidic organic buffer solution.

17. The method of claim 12, wherein the at least one C6 and/or C5 sugar in the first aqueous solution comprises solubilized C6 and/or C5 sugar, or suspended C6 and/or C5 sugar particulate, or any combinations thereof.

18. The method of claim 12, wherein the enzymatically digesting of the comminuted lignocellulosic material in the first aqueous solution with the first enzyme blend comprises agitating the first mixture for at least about 1 hour at a temperature of from about 30° C. to about 60° C., and the separating comprises discontinuing the agitating and decanting the at least a portion of the first aqueous solution containing the at least one C6 and/or C5 sugar from the first insoluble portion comprising settled solids.

19. The method of claim 12, wherein the lignocellulosic material is cotton seed hulls, grain hulls, sugarcane bagasse, corn stover, corn cobs, straw, switchgrass, leaves, stalks, plant shells, softwood pieces, hardwood pieces, sawdust, papermill pulp, waste paper, recycled paper, or any combinations thereof.

20. The method of claim 12, wherein the lignocellulosic material comprises cotton seed hulls.

21. The method of claim 12, wherein the first enzyme blend comprises at least one enzyme from at least two of groups 1) to 5):
1) endoglucanase, carboxymethylcellulase;
2) exoglucanase, avicelase;
3) cellobiase, beta-glucosidase, alpha-glucosidase;
4) endo 1,4-beta-xylanase, endo-(1,4)-beta xylanohydrolase;
5) beta-1,3-xylanase, 1,3-beta-D-xylosidase, and exo-1,3-beta-xylosidase.

22. The method of claim 12, wherein the second enzyme blend comprises at least one enzyme from at least two of groups 1a) to 20a):
1a) endo-1,3(4)-beta-glucanase;
2a) laminarinase (endo-1,3-beta-glucanase);
3a) exo-1,2-1,6-alpha-mannosidase;
4a) beta-D-xylopyranosyl-(1,4)-beta-D-xylopyranose;
5a) alpha-N-arabinofuranosidase;
6a) feruloyl esterase;
7a) endo-1,5-alpha-arabinanase;
8a) pectinase;
9a) polygalacturonase;
10a) pectin esterase;
11a) aspartic protease;
12a) metallo protease;
13a) endo-(1,4)-mannanase;
14a) phytase;
15a) alpha-glucuronidase and beta-glucuronidase;
16a) hexenuronidase;
17a) alkaline phosphatase and acid phosphatase;
18a) alpha-galactosidase and beta-galactosidase;
19a) beta-mannosidase;
20a) alpha-fucosidase.

23. The method of claim 12, wherein yield of lignin is at least about 90%.

24. The method of claim 12, wherein yield of the at least one C6 and/or C5 sugar is at least about 50% based on the original amount present in the lignocellulosic material.

25. The method of claim 12, further comprising biofermentation processing of the at least one C6 and/or C5 sugar.

26. A system for isolating components of lignocellulosic material comprising:
at least one comminutor for comminuting lignocellulosic material;
at least one digestor comprising a vessel equipped with an internal agitator, wherein the vessel is operable for holding a mixture of the comminuted lignocellulosic material, at least one enzyme blend, and aqueous solution in the absence of any prior non-enzymatic lignin separation treatment of the lignocellulosic material, and operable for enzymatically digesting the mixture with agitation for enzymatic hydrolysis liberating at least one C6 and/or C5 sugar from an insoluble portion of the lignocellulosic material comprising lignin; and
a separator for separating at least a portion of the aqueous solution containing the at least one sugar from the insoluble portion comprising lignin.

27. The system of claim 26, further comprising:
a dryer for drying the insoluble portion comprising lignin;
a comminutor for comminuting the first insoluble material;
a digestor comprising a vessel equipped with an internal agitator, wherein the vessel is operable for holding a mixture of the comminuted first insoluble material, at least one enzyme, and aqueous solution, and contacting the mixture with agitation for enzymatic hydrolysis liberating at least one C6 and/or C5 sugar from an insoluble portion of the lignocellulosic material comprising lignin; and
a separator for separating at least a portion of the aqueous solution containing the at least one sugar from the insoluble portion comprising lignin.

28. The system of claim 26, further comprising a biofermentation processing system adapted to receive the liberated C6 and/or C5 sugar from the digestor and ferment the C6 and/or C5 sugar into ethanol.
